(19) United States
(12) Patent Application Publication
(10) Pub. No.: US 2010/0311805 A1
(43) Pub. Date: Dec. 9, 2010

(54) COMBINATION OF HISTONE DEACETYLASE INHIBITORS AND RADIATION

(75) Inventor: Richard William Versace, Wanaque, NJ (US)

Correspondence Address: NOVARTIS;CORPORATE INTELLECTUAL PROPERTY, ONE HEALTH PLAZA 101/2, EAST HANOVER, NJ, 07936-1080, US

(73) Assignee: NOVARTIS AG, Basel (CH)

(21) Appl. No.: 12/852,770

(22) Filed: Aug. 9, 2010

Related U.S. Application Data
(63) Continuation of application No. 12/089,658, filed on Apr. 9, 2008.
(60) Provisional application No. 60/729,783, filed on Oct. 24, 2005.

(51) Int. Cl. A61K 31/404; A61P 35/00

(52) U.S. Cl. 514/419

(57) ABSTRACT

This invention relates to organic compounds of formula (I):

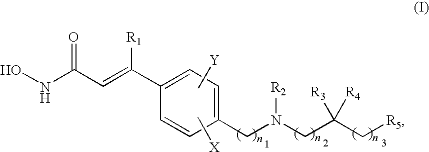

in particular, to pharmaceutical compositions for use in combination with ionizing radiation for the delay of progression or treatment of a proliferative disease, especially a solid tumor disease.

This is a continuation of application Ser. No. 12/089,658 filed on Apr. 9, 2008, which is a National Stage of International Application No. PCT/US06/41567 filed on Oct. 23, 2006, which claims benefit of U.S. Provisional Application No. 60/729,783 filed Oct. 24, 2005, which in its entirety are herein incorporated by reference.

### FIELD OF INVENTION

This invention relates to organic compounds, in particular, to pharmaceutical compositions for use in combination with ionizing radiation for the delay of progression or treatment of a proliferative disease, especially a solid tumor disease.

### SUMMARY OF THE INVENTION

We have now found that certain histone deacetylase inhibitors, i.e., HDACs, are effective when used in combination with ionizing radiation for the delay of progression or treatment of a proliferative disease, especially a solid tumor disease.

### DETAILED DESCRIPTION OF THE DRAWINGS

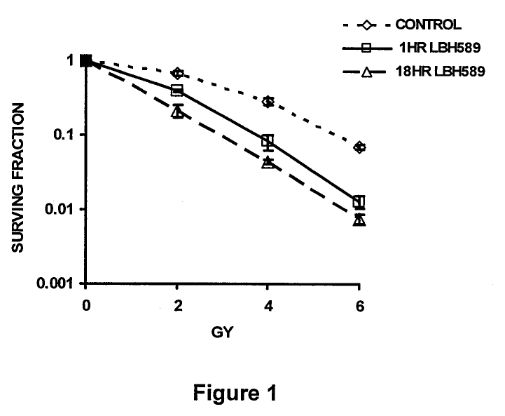

FIG. 1 illustrates the mean surviving fraction and standard error for each treatment using LBH589 and 0-6 Gy on clonogenic analysis of H460 cell lines.

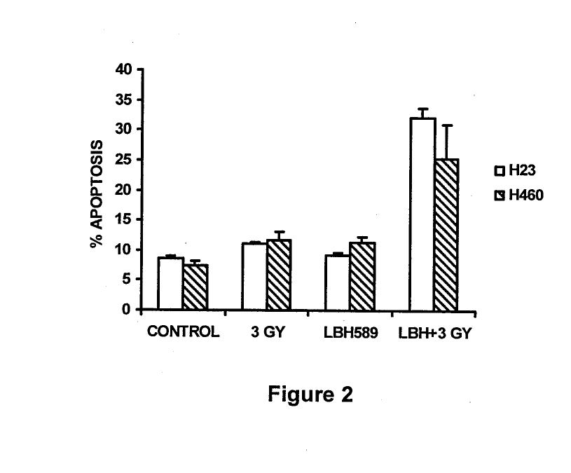

FIG. 2 illustrates the results from Annexin V-FITC/PI flow cytometry analysis of the apoptosis effect of HDAC inhibition by LBH589.

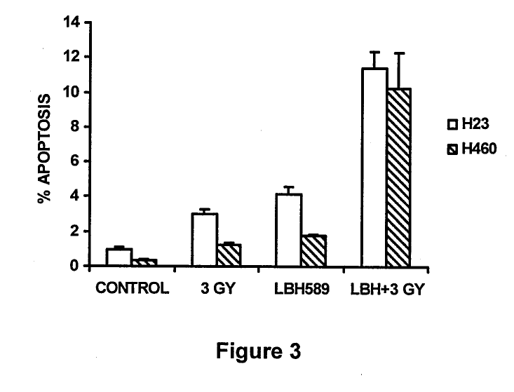

FIG. 3 illustrates the mean percentage and standard error of pyknotic nuclei determined by DAPI staining to confirm the ability of LBH589 to sensitize human lung cancer cell lines.

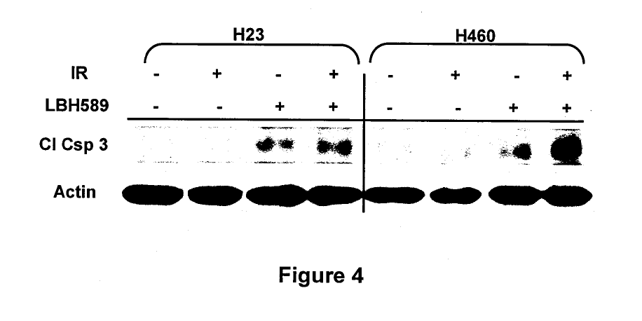

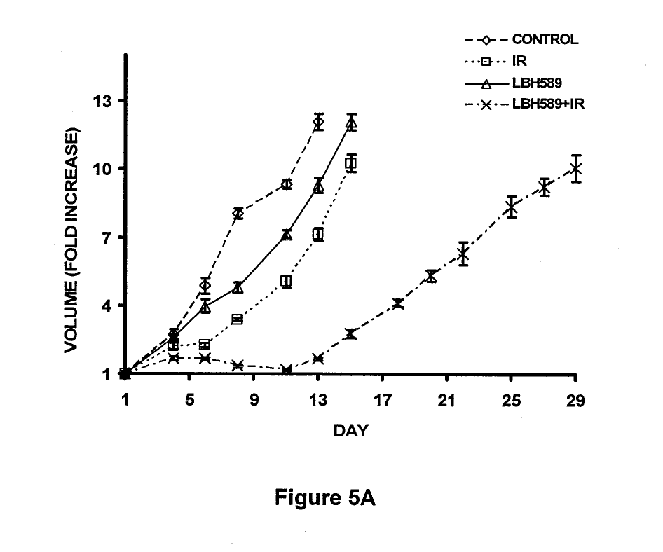

FIG. 4 illustrates the Western immunoblots for cleaved caspase 3 and actin. LBH589 induced caspase 3 cleavage to verify the role of apoptosis in cells treated with LBH589 and radiation.

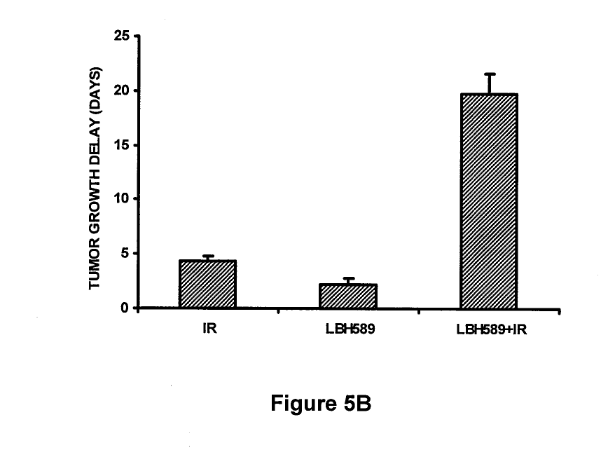

FIG. 5 illustrates the fold increase in tumor volume (A) and the tumor growth delay (B) for each treatment group with LBH589.

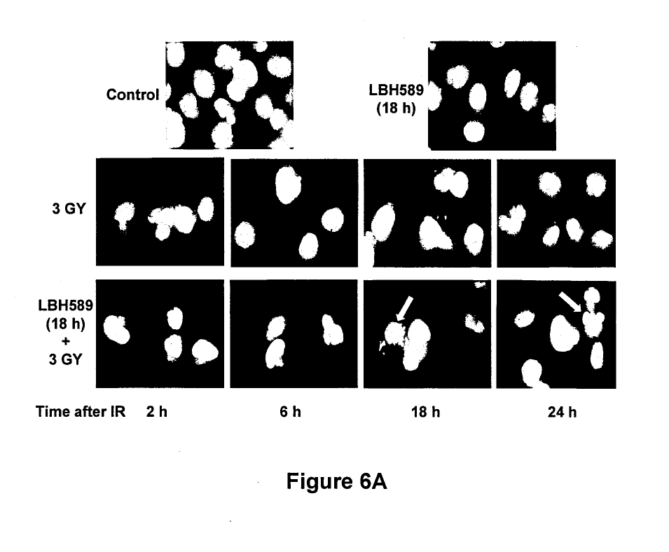

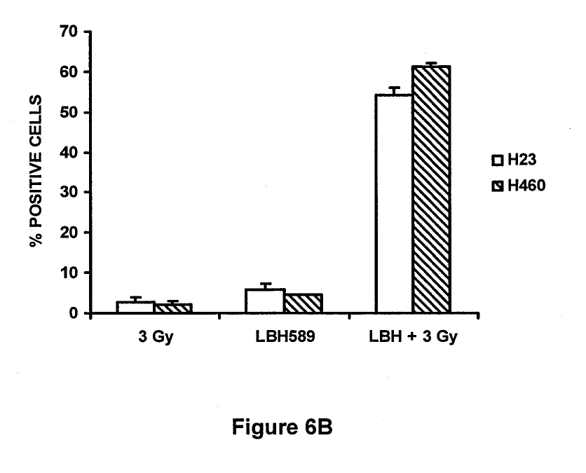

FIG. 6A illustrates representative photographs of the H23 cell line treated with combinations of LBH589 and IR.

FIG. 6B illustrates the number of γ-H2AX foci present 24 hrs after IR.

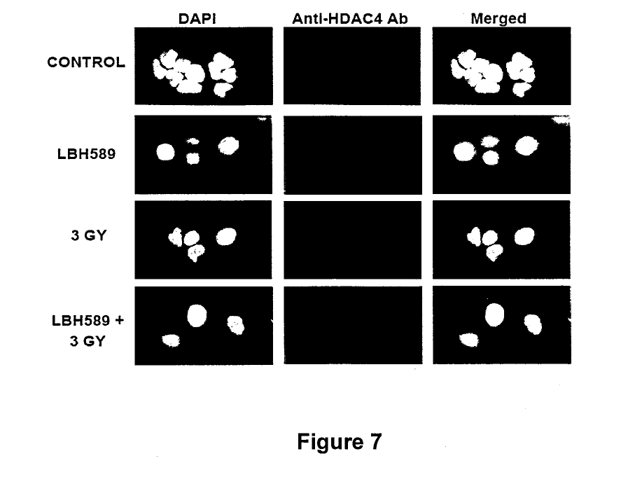

FIG. 7 illustrates representative photographs of the H460 cell line probed with anti-HDAC 4 antibodies and rhodamine labeled secondary antibodies then counterstained with DAPI.

### DETAILED DESCRIPTION OF THE INVENTION

Accordingly, the invention provides a method for the delay of progression or treatment of a proliferative disease, especially a solid tumor disease in a subject in need of such treatment which comprises administering to the subject an effective amount of an HDAC of formula (I):

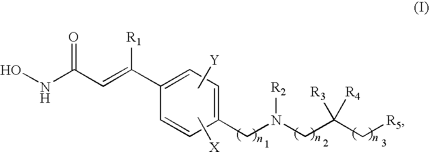

wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₆alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and —(CH₂)ₙR₇;
R₃ and R₄ are the same or different and, independently, H; C₁-C₆alkyl; acyl; or acylamino, or
R₃ and R₄, together with the carbon to which they are bound, represent C═O, C═S or C═NR₈; or
R₂, together with the nitrogen to which it is bound, and R₃, together with the carbon to which it is bound, can form a C₄-C₉heterocycloalkyl; a heteroaryl; a polyheteroaryl; a non-aromatic polyheterocycle; or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅; SR₁₅; S(O)R₁₆; SO₂R₁₇; NR₁₃R₁₄; and NR₁₂SO₂R₆;
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H; C₁-C₄alkyl; and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl, or
R₁₃ and R₁₄, together with the nitrogen to which they are bound, are C₄-C₉heterocycloalkyl; heteroaryl; polyheteroaryl; non-aromatic polyheterocycle; or mixed aryl and non-aryl polyheterocycle;
R₁₅ is selected from H; C₁-C₆alkyl; C₄-C₉cycloalkyl; C₄-C₉heterocycloalkyl; aryl; heteroaryl; arylalkyl; heteroarylalkyl; and (CH₂)ₘZR₁₂;
R₁₆ is selected from C₁-C₆alkyl; C₄-C₉cycloalkyl; C₄-C₉heterocycloalkyl; aryl; heteroaryl; polyheteroaryl; arylalkyl; heteroarylalkyl; and (CH₂)ₘZR₁₂;
R₁₇ is selected from C₁-C₆alkyl; C₄-C₉cycloalkyl; C₄-C₉heterocycloalkyl; aryl; aromatic polycycles; heteroaryl; arylalkyl; heteroarylalkyl; polyheteroaryl and NR₁₃R₁₄;
m is an integer selected from 0-6; and
Z is selected from 0; NR₁₃; S; and S(O),
   or a pharmaceutically acceptable salt thereof.

As appropriate, “unsubstituted” means that there is no substituent or that the only substituents are hydrogen.

Halo substituents are selected from fluoro, chloro, bromo and iodo, preferably fluoro or chloro.

Alkyl substituents include straight- and branched-C₁-C₆alkyl, unless otherwise noted. Examples of suitable straight- and branched-C₁-C₆alkyl substituents include methyl, ethyl, n-propyl, 2-propyl, n-butyl, sec-butyl, t-butyl and the like. Unless otherwise noted, the alkyl substituents include both unsubstituted alkyl groups and alkyl groups that are substituted by one or more suitable substituents, including unsaturation, i.e., there are one or more double or triple C—C bonds; acyl; cycloalkyl; halo; oxyalkyl; alkylamino; aminoalkyl; acylamino; and OR₁₅, e.g., alkoxy. Preferred substituents for alkyl groups include halo, hydroxy, alkoxy, oxyalkyl, alkylamino and aminoalkyl.

Cycloalkyl substituents include C₃-C₉cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆alkyl, halo, hydroxy, aminoalkyl, oxyalkyl, alkylamino and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as, without limitation, alkoxy, alkyl amines, alkyl ketones, arylalkyl, heteroarylalkyl, alkylsulfonyl and alkyl ester substituents and the like.

Heterocycloalkyl substituents include 3- to 9-membered aliphatic rings, such as 4- to 7-membered aliphatic rings, containing from 1-3 heteroatoms selected from nitrogen, sulfur, oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane and 1,4-oxathiapane. Unless otherwise noted, the rings are unsubstituted or substituted on the carbon atoms by one or more suitable substituents, including C₁-C₆alkyl; C₄-C₉cycloalkyl; aryl; heteroaryl; arylalkyl, e.g., benzyl; heteroarylalkyl, e.g., pyridylmethyl; halo; amino; alkyl amino and OR₁₅, e.g., alkoxy. Unless otherwise noted, nitrogen heteroatoms are unsubstituted or substituted by H, C₁-C₄alkyl; arylalkyl, e.g., benzyl; heteroarylalkyl, e.g., pyridylmethyl; acyl; aminoacyl; alkylsulfonyl; and arylsulfonyl.

Cycloalkylalkyl substituents include compounds of the formula —(CH₂)ₙ₅-cycloalkyl, wherein n₅ is a number from 1-6. Suitable alkylcycloalkyl substituents include cyclopentylmethyl, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents including C₁-C₆alkyl; cycloalkylalkyl, e.g., cyclopropylmethyl; O(CO)alkyl; oxyalkyl; halo; nitro; amino; alkylamino; aminoalkyl; alkyl ketones; nitrile; carboxyalkyl; alkylsulfonyl; aminosulfonyl; arylsulfonyl and OR₁₅, such as alkoxy. Preferred substituents include including C₁-C₆alkyl; cycloalkyl, e.g., cyclopropylmethyl; alkoxy; oxyalkyl; halo; nitro; amino; alkylamino; aminoalkyl; alkyl ketones; nitrile; carboxyalkyl; alkylsulfonyl; arylsulfonyl and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents including C₁-C₆alkyl; alkylcycloalkyl, e.g., cyclopropylmethyl; oxyalkyl; halo; nitro; amino; alkylamino; aminoalkyl; alkyl ketones; nitrile; carboxyalkyl; alkylsulfonyl; arylsulfonyl; aminosulfonyl and OR₁₅, such as alkoxy.

Heteroaryl substituents include compounds with a 5- to 7-membered aromatic ring containing one or more heteroatoms, e.g., from 1-4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, e.g., by R₁₃, especially useful N substituents include H, C₁-C₄alkyl, acyl, aminoacyl and sulfonyl.

Arylalkyl substituents include groups of the formula —(CH₂)ₙ₅-aryl, —(CH₂)ₙ₅₋₁—(CH-aryl)-(CH₂)ₙ₅-aryl or —(CH₂)ₙ₅₋₁CH(aryl)(aryl), wherein aryl and n5 are defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl, wherein heteroaryl and ₙ₅ are defined above and the bridging group is linked to a carbon or a nitrogen of the heteroaryl portion, such as 2-, 3- or 4-pyridylmethyl, imidazolylmethyl, quinolylethyl and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NR₁₃R₁₄)—(CH₂)ₙ—R₅, wherein n, R₁₃, R₁₄ and R₅ are described above. Suitable aminoacyl substituents include natural and non-natural amino acids, such as glycinyl, D-tryptophanyl, L-lysinyl, D- or L-homoserinyl, 4-aminobutryic acyl and ±−3-amin-4-hexenoyl.

Non-aromatic polycycle substituents include bicyclic and tricyclic fused ring systems where each ring can be 4- to 9-membered and each ring can contain zero, one or more double and/or triple bonds. Suitable examples of non-aromatic polycycles include decalin, octahydroindene, perhydrobenzocycloheptene and perhydrobenzo-[f]-azulene. Such substituents are unsubstituted or substituted as described above for cycloalkyl groups.

Mixed aryl and non-aryl polycycle substituents include bicyclic and tricyclic fused ring systems where each ring can be 4- to 9-membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene and 9H-fluorene. Such substituents are unsubstituted or substituted by nitro or as described above for cycloalkyl groups.

Polyheteroaryl substituents include bicyclic and tricyclic fused ring systems where each ring can independently be 5- or 6-membered and contain one or more heteroatom, for example, 1, 2, 3 or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂))₁₋₃H. Nitrogen atoms are unsubstituted or substituted, e.g., by R₁₃, especially useful N substituents include H, C₁-C₄alkyl, acyl, aminoacyl and sulfonyl.

Non-aromatic polyheterocyclic substituents include bicyclic and tricyclic fused ring systems where each ring can be 4- to 9-membered, contain one or more heteroatom, e.g., 1, 2, 3 or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, e.g., by R₁₃, especially useful N substituents include H, C₁-C₄alkyl, acyl, aminoacyl and sulfonyl.

Mixed aryl and non-aryl polyheterocycles substituents include bicyclic and tricyclic fused ring systems where each ring can be 4- to 9-membered, contain one or more heteroatom chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents including —N—OH, ═N—OH, alkyl and the alkyl substituents identified above. Nitrogen atoms are unsubstituted or substituted, e.g., by R₁₃; especially useful N substituents include H, C₁-C₄alkyl, acyl, aminoacyl and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, e.g., methane sulfonyl, benzene sulfonyl, tosyl and the like.

Acyl substituents include groups of formula —C(O)—W, —OC(O)—W, —C(O)—O—W or —C(O)NR₁₃R₁₄, where W is R₁₆, H or cycloalkylalkyl.

Acylamino substituents include substituents of the formula —N(R₁₂)C(O)—W, —N(R₁₂)C(O)—O—W and —N(R₁₂)C(O)—NHOH and R₁₂ and W are defined above.

The R₂ substituent HON—C(O)—CH═C(R₁)-aryl-alkyl- is a group of the formula

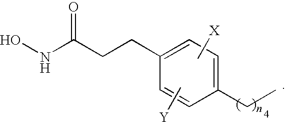

Preferences for each of the substituents include the following:
R₁ is H, halo or a straight-chain C₁-C₄alkyl;
R₂ is selected from H, C₁-C₆alkyl, C₄-C₉cycloalkyl, C₄-C₉heterocycloalkyl, aryl, heteroaryl, arylalkyl, heteroarylalkyl, —(CH₂)ₙC(O)R₆, amino acyl and —(CH₂)ₙR₇;
R₃ and R₄ are the same or different and independently selected from H and C₁-C₆alkyl, or
R₃ and R₄, together with the carbon to which they are bound, represent C═O, C═S or C═NR₅;
R₅ is selected from H, C₁-C₆alkyl, C₄-C₉cycloalkyl, C₄-C₉heterocycloalkyl, aryl, heteroaryl, arylalkyl, heteroarylalkyl, a aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n₁ is 1-6, each carbon atom is unsubstituted or independently substituted with R₃ and/or R₄;
X and Y are the same or different and independently selected from H, halo, C₁-C₄alkyl, CF₃, NO₂, C(O)R₁, OR₉, SR₉, CN and NR₁₀R₁₁;
R₆ is selected from H, C₁-C₆alkyl, C₄-C₉cycloalkyl, C₄-C₉heterocycloalkyl, alkylcycloalkyl, aryl, heteroaryl, arylalkyl, heteroarylalkyl, OR₁₂ and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H, OR₁₅, NR₁₃R₁₄, C₁-C₆alkyl, C₄-C₉cycloalkyl, C₄-C₉heterocycloalkyl, aryl, heteroaryl, arylalkyl and heteroarylalkyl;
R₉ is selected from C₁-C₄alkyl and C(O)-alkyl;
R₁₀ and R₁₁ are the same or different and independently selected from H, C₁-C₄alkyl and —C(O)-alkyl;
R₁₂ is selected from H, C₁-C₆alkyl, C₄-C₉cycloalkyl, C₄-C₉heterocycloalkyl, aryl, heteroaryl, arylalkyl and heteroarylalkyl;
R₁₃ and R₁₄ are the same or different and independently selected from H, C₁-C₆alkyl, C₄-C₉cycloalkyl, C₄-C₉heterocycloalkyl, aryl, heteroaryl, arylalkyl, heteroarylalkyl and amino acyl;
R₁₅ is selected from H, C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, heteroaryl, arylalkyl, heteroarylalkyl and NR₁₃R₁₄;
m is an integer selected from 0-6; and
Z is selected from O, NR₁₃, S and S(O);
   or a pharmaceutically acceptable salt thereof.

Useful compounds of the formula (I), include those wherein each of R₁, X, Y, R₃ and R₄ is H, including those wherein one of n₂ and n₃ is 0 and the other is 1, especially those wherein R₂ is H or —CH₂—CH₂—OH.

One suitable genus of hydroxamate compounds are those of formula (Ia):

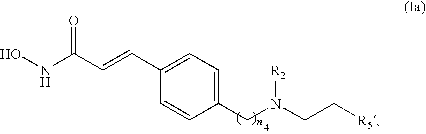

wherein
n₄ is 0-3;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇; and
R₅ is heteroaryl; heteroarylalkyl, e.g., pyridylmethyl; aromatic polycycles; non-aromatic polycycles; mixed aryl and non-aryl polycycles; polyheteroaryl or mixed aryl; and non-aryl polyheterocycles;
   or a pharmaceutically acceptable salt thereof.

Another suitable genus of hydroxamate compounds are those of formula (Ia):

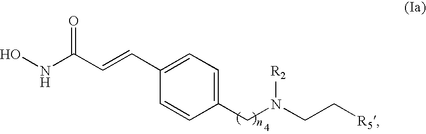

wherein
n₄ is 0-3;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₅ is aryl; arylalkyl; aromatic polycycles; non-aromatic polycycles and mixed aryl; and non-aryl polycycles, especially aryl, such as p-fluorophenyl, p-chlorophenyl, p-O—C₁-C₄alkylphenyl, such as p-methoxyphenyl, and p-C₁-C₄alkylphenyl; and arylalkyl, such as benzyl, ortho-, meta- or para-fluorobenzyl, ortho-, meta- or para-chlorobenzyl, ortho-, meta- or para-mono, di- or tri-O—C₁-C₄alkylbenzyl, such as ortho-, meta- or para-methoxybenzyl, m,p-diethoxybenzyl, o,m,p-triimethoxybenzyl and ortho-, meta- or para-mono, di- or tri-C₁-C₄alkylphenyl, such as p-methyl, m,m-diethylphenyl;
   or a pharmaceutically acceptable salt thereof.

Another interesting genus is the compounds of formula (Ib):

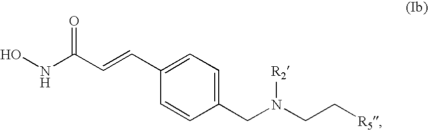

wherein
R2 is selected from H; C₁-C₆alkyl; C₄-C₆cycloalkyl; cycloalkylalkyl, e.g., cyclopropylmethyl; (CH₂)₂₋₄OR₂₁, where R₂₁ is H, methyl, ethyl, propyl and i-propyl; and
R₅″ is unsubstituted 1H-indol-3-yl, benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl;
   or a pharmaceutically acceptable salt thereof.

Another interesting genus of hydroxamate compounds are the compounds of formula (Ic):

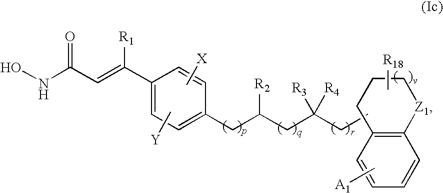

wherein
the ring containing Z₁ is aromatic or non-aromatic, which non-aromatic rings are saturated or unsaturated,
Z₁ is O, S or N—R₂₀;
R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl, such as 2-furanyl, 2-thiophenyl or 2-, 3- or 4-pyridyl;
R₂₀ is H; C₁-C₆alkyl; C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl and toluenesulfonyl;
A₁ is 1, 2 or 3 substituents which are independently H; C₁-C₆alkyl; —OR₁₉; halo; alkylamino; aminoalkyl; halo; or heteroarylalkyl, e.g., pyridylmethyl;
R₁₉ is selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl and —(CH₂CH═CH(CH₃)(CH₂))₁₋₃H;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
v is 0, 1 or 2;
p is 0-3; and
q is 1-5 and r is 0, or
q is 0 and r is 1-5;
   or a pharmaceutically acceptable salt thereof. The other variable substituents are as defined above.

Especially useful compounds of formula (Ic), are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3, especially those wherein Z₁ is N—R₂₀. Among these compounds R₂ is preferably H or —CH₂—CH₂—OH and the sum of q and r is preferably 1.

Another interesting genus of hydroxamate compounds are the compounds of formula (Id):

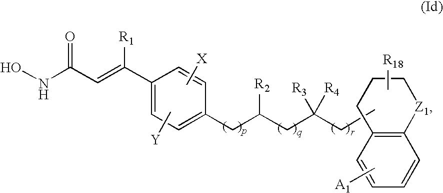

wherein
Z₁ is O, S or N—R₂₀;
R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl;
R₂₀ is H; C₁-C₆alkyl, C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl, toluenesulfonyl);
A₁ is 1, 2 or 3 substituents which are independently H, C₁-C₆alkyl, —OR₁₉ or halo;
R₁₉ is selected from H; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
p is 0-3; and
q is 1-5 and r is 0, or
q is 0 and r is 1-5;
   or a pharmaceutically acceptable salt thereof. The other variable substituents are as defined above.

Especially useful compounds of formula (Id), are those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

The present invention further relates to compounds of the formula (Ie):

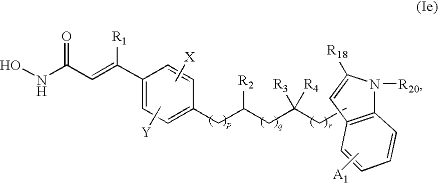

or a pharmaceutically acceptable salt thereof. The variable substituents are as defined above.

Especially useful compounds of formula (Ie), are those wherein R₁₈ is H, fluoro, chloro, bromo, a C₁-C₄alkyl group, a substituted C₁-C₄alkyl group, a C₃-C₇cycloalkyl group, unsubstituted phenyl, phenyl substituted in the para position, or a heteroaryl, e.g., pyridyl, ring.

Another group of useful compounds of formula (Ie), are those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1. Among these compounds p is preferably 1 and R₃ and R₄ are preferably H.

Another group of useful compounds of formula (Ie), are those wherein R₁₈ is H, methyl, ethyl, t-butyl, trifluoromethyl, cyclohexyl, phenyl, 4-methoxyphenyl, 4-trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H or —(CH₂)ₚCH₂OH, wherein p is 1-3; especially those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula (Ie), wherein R₂₀ is H or C₁-C₆alkyl, especially H, are important members of each of the subgenuses of compounds of formula (Ie) described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide or a pharmaceutically acceptable salt thereof, are important compounds of formula (Ie).

The present invention further relates to the compounds of the formula (If):

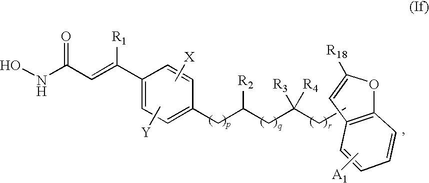

or a pharmaceutically acceptable salt thereof. The variable substituents are as defined above.

Useful compounds of formula (If), are include those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

N-hydroxy-3-[4-[[[2-(benzofur-3-yl)-ethyl]-amino]methyl]phenyl]-2E-2-propenamide or a pharmaceutically acceptable salt thereof, is an important compound of formula (If).

The compounds described above are often used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts and amino acid addition salts and sulfonate salts. Acid addition salts include inorganic acid addition salts, such as hydrochloride, sulfate and phosphate; and organic acid addition salts, such as alkyl sulfonate, arylsulfonate, acetate, maleate, fumarate, tartrate, citrate and lactate. Examples of metal salts are alkali metal salts, such as lithium salt, sodium salt and potassium salt; alkaline earth metal salts, such as magnesium salt and calcium salt, aluminum salt and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

Additional HDAI compounds within the scope of formula (I), and their synthesis, are disclosed in WO 02/22577 published Mar. 21, 2002 which is incorporated herein by reference in its entirety. Two preferred compounds within the scope of WO 02/22577 are N-hydroxy-3-[4-[(2-hydroxymethyl){2-(1H-indol-3-yl)ethyl]-amino]methyl]phenyl]-2E-2-propenamide, of formula (II):

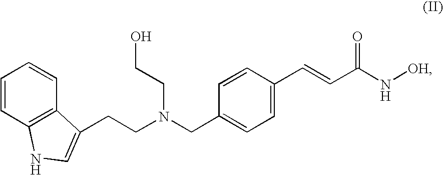

or a pharmaceutically acceptable salt thereof, and
N-hydroxy-3-[4-[[[2-(2-methyl-1H-indol-3-yl)-ethyl]-amino]methyl]phenyl]-2E-2-propenamide (LBH589), of formula (III):

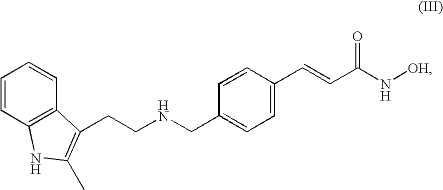

or a pharmaceutically acceptable salt thereof.

Further, the invention provides the use of a compound of formula (I), or pharmaceutically acceptable salt or prodrug ester thereof, for the preparation of a medicament for use in combination with ionizing radiation in the treatment of a proliferative disease.

In a further aspect, the invention provides use of an HDAC of formula (I), or pharmaceutically acceptable salt or prodrug ester thereof, in combination with ionizing radiation for the treatment of a proliferative disease, especially a solid tumor.

In yet further aspect, the invention provides an HDAC of formula (I), or pharmaceutically acceptable salt or prodrug ester thereof, as active ingredient for use in combination with ionizing radiation for the treatment of a proliferative disease, especially a solid tumor.

In still yet further aspect, the invention provides a package comprising an HDAC of formula (I), or pharmaceutically acceptable salt or prodrug ester thereof, together with instructions for the use In combination with ionizing radiation for the treatment of a proliferative disease, especially a solid tumor.

The term “delay of progression”, as used herein, means administration of the combination to patients being in an early phase of the proliferative disease to be treated.

The term “solid tumor disease”, as used herein, comprises, but is not restricted to glioma, thyroid cancer, breast cancer, ovarian cancer, cancer of the colon and generally the GI tract, cervix cancer, lung cancer, in particular, small-cell lung cancer, and non-small-cell lung cancer, head and neck cancer, bladder cancer, cancer of the prostate or Kaposi's sarcoma. In one preferred embodiment of the invention, the tumor disease to be treated is glioma, cancer of the prostate or thyroid cancer. The present combination inhibits the growth of solid tumors, but also liquid tumors. Furthermore, depending on the tumor type and the particular combination used, a decrease of the tumor volume can be obtained. The combinations disclosed herein are also suited to prevent the metastatic spread of tumors and the growth or development of micrometastases.

Combination refers to administration of an amount of HDAC of formula (I) in combination with administration of an amount of ionizing radiation such that there is a synergistic effect which would not be obtained if an HDAC of formula (I) is administered without separate, simultaneous or sequential administration of ionizing radiation. Wherein administration of ionizing radiation can be continuous, sequential or sporadic. Or an effect which would not be obtained if there is administered ionizing radiation without the separate, simultaneous or sequential administration of an HDAC derivative of formula (I), wherein administration can be continuous, sequential or sporadic.

Preferably combination refers to administration of an amount of HDAC of formula (I) in combination with administration of an amount of ionizing radiation such that there is a synergistic antiproliferative effect and/or a clonogenic cell killing effect that would not be obtained if:

1) The HDAC of formula (I) is administered without prior, simultaneous or subsequent administration of ionizing radiation, wherein administration can be continuous, sequential or sporadic;
2) There is administration of ionizing radiation without the prior, simultaneous or subsequent administration of an HDAC of formula (I), wherein administration can be continuous, sequential or sporadic.

The term “ionizing radiation”, referred to above and hereinafter, means ionizing radiation that occurs as either electromagnetic rays (such as X-rays and gamma rays) or particles (such as alpha and beta particles). Ionising radiation is provided in, but not limited to, radiation therapy and is known in the art [see Hellman, Principles of Radiation Therapy, Cancer, in Principles and Practice of Oncology, pp. 248-275, Devita et al., Ed., 4^{th} Edition, Vol. 1 (1993)].

A combination which comprises:
(a) an HDAC of formula (I), which may be present in free form or in the form of a pharmaceutically acceptable salt and optionally at least one pharmaceutically acceptable carrier; and
(b) ionizing radiation, will be referred to hereinafter as a COMBINATION OF THE INVENTION.

The nature of proliferative diseases like solid tumor diseases is multifactorial. Under certain circumstances, drugs with different mechanisms of action may be combined. However, just considering any combination of drugs having different mode of action does not necessarily lead to combinations with advantageous effects.

In the combination of the invention, HDACs of formula (I), and pharmaceutically acceptable salts and prodrug derivatives, are preferably used in the form of pharmaceutical preparations that contain the relevant therapeutically effective amount of active ingredient optionally together with or in admixture with inorganic or organic, solid or liquid, pharmaceutically acceptable carriers which are suitable for administration.

In an alternative embodiment, the ionizing radiation is given as a pre-treatment, i.e., before the treatment with the COMBINATION OF THE INVENTION is started; the ionizing radiation alone is administered to the patient for a defined period of time, e.g., daily administration of the ionizing radiation alone for two or three days or weeks.

The HDAC pharmaceutical compositions may be, for example, compositions for enteral, such as oral, rectal, aerosol inhalation or nasal administration, compositions for parenteral, such as intravenous or subcutaneous administration, or compositions for transdermal administration (e.g., passive or iontophoretic), or compositions for topical administration.

Preferably, the HDAC pharmaceutical compositions are adapted to oral administration.

The pharmaceutical compositions according to the invention can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carries, especially suitable for enteral or parenteral application.

The novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example, by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In preparing the compositions for oral dosage form, any of the usual pharmaceutical media may be employed, such as, for example, water, glycols, oils, alcohols, flavouring agents, preservatives, colouring agents; or carriers, such as starches, sugars, microcrystalline cellulose, diluents, granulating agents, lubricants, binders, disintegrating agents and the like in the case of oral solid preparations, such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed.

In particular, a therapeutically effective amount of each combination partner of the COMBINATION OF THE INVENTION may be administered simultaneously or sequentially and in any order, and the components may be administered separately or as a fixed combination. For example, the method of delay of progression or treatment of a proliferative disease according to the invention may comprise:
(i) administration of the first combination partner; and
(ii) administration of the second combination partner,
   wherein administration of a combination partner may be simultaneous or sequential in any order, in jointly therapeutically effective amounts, preferably in synergistically effective amounts, e.g., in daily or weekly dosages corresponding to the amounts described herein. The individual combination partners of the COMBINATION OF THE INVENTION can be administered separately at different times during the course of therapy or concurrently. Furthermore, the term administering also encompasses the use of a pro-drug of an HDAC of formula (I) that converts in vivo to the combination partner as such. The instant invention is therefore to be understood as embracing all such regimes of simultaneous or alternating treatment and the term “administering” is to be interpreted accordingly.

The dosage of ionizing radiation and an HDAC of formula (I) in relation to each other is preferably in a ratio that is synergistic.

If the warm-blooded animal is a human, the dosage of a compound of formula (I) is preferably an appropriate dose in the range from 100-1,500 mg daily, e.g., 200-1,000 mg/day, such as 200, 400, 500, 600, 800, 900 or 1,000 mg/day, administered in one or two doses daily. Appropriate dosages and the frequency of administration of the death receptor ligand will depend on such factors, as the nature and severity of the indication being treated, the desired response, the condition of the patient and so forth.

The particular mode of administration and the dosage of a compound of formula (I) may be selected by the attending physician taking into account the particulars of the patient, especially age, weight, life style, activity level, etc.

The dosage of an HDAC of formula (I) may depend on various factors, such as effectiveness and duration of action of the active ingredient, mode of administration, effectiveness and duration of action of the ionizing radiation and/or sex, age, weight and individual condition of the subject to be treated.

The dosage of ionizing radiation may depend on various factors, such as effectiveness and duration of action of the ionizing radiation, mode of administration, location of administration, effectiveness and duration of action of the HDAC of formula (I) and/or sex, age, weight and individual condition of the subject to be treated. The dosage of ionizing radiation is generally defined in terms of radiation absorbed dose, time and fraction, and must be carefully defined by the attending physician.

In one preferred embodiment of the invention the combination comprises ionizing radiation and hydroxy-3-[4-[[[2-(2-methyl-1H-indol-3-yl)-ethyl]-amino]methyl]phenyl]-2E-2-propenamide, of formula (III) above or a pharmaceutically acceptable salt thereof.

Moreover, the present invention relates to a method of treating a warm-blooded animal having a proliferative disease comprising administering to the animal a COMBINATION OF THE INVENTION in a way that is jointly therapeutically effective against a proliferative disease and in which the combination partners can also be present in the form of their pharmaceutically acceptable salts.

Furthermore, the present invention pertains to the use of a COMBINATION OF THE INVENTION for the delay of progression or treatment of a proliferative disease and for the preparation of a medicament for the delay of progression or treatment of a proliferative disease.

The following example is merely illustrative and not meant to limit the scope of the present invention in any manner:

### Example 1

Tumor Model. LLC, H450 and H23 cell lines are obtained from ATTC. These cell lines form tumors in nude mice following s.c. injection into either hind limb. Cells are trypsinized and counted by hemocytometer. Cells are washed in complete medium, and 10⁶ cells will be injected s.c. into the hind limb or into the dorsal skin fold window.

Western Immunoblots. LLC, H450 and H23 cells are serum starved overnight in DMEM/F-12 media (Gibco). Cells are then treated with 10 μM of Compound III for 1 hour and/or irradiated with 3 Gy. Cells are washed twice with PBS and lysis buffer (20 nM Tris, 150 mM NaCl, 1 mM EDTA, 1% Triton X-100, 2.5 mM Na PP, 1 mM phenylmethylsulfonyl fluoride, and 1 μg/mL leupeptin) are added. Protein concentration are quantified by the Bio-Rad method. Twenty (20) μg of total protein are loaded into each well and separated by 7% or 10% SDS-PAGE gel, depending on the size of the target protein being investigated. The proteins are transferred onto nitrocellulose membranes (Hybond ECL; Amersham, Arlington Heights, Ill.) and probed with primary antibodies to caspase3, cleaved caspase3, phospho-Akt, Akt, PDGFR α and β (Cell Signaling; 1:1000). Blots are washed and probed with goat anti-rabbit secondary antibody (Sigma; 1:1000).

Apoptosis Quantification. Morphologic analysis of apoptosis in LLC cells are performed under microscope using propidium iodide staining. Apoptotic cells are identified according to their nuclear condensation and fragmentation. Briefly, LLC Cells are treated with 3 Gy and N-hydroxy-3-[4-[[[2-(2-methyl-1H-indol-3-yl)-ethyl]-amino]methyl]phenyl]-2E-2-propenamide (LBH589) (100 nM) or both agents. After 24 hours, cells are washed with PBS several times, permeabilized with 30% methanol and stained with propidium iodide in PBS. Apoptotic and non-apoptotic cells are counted in multiple randomly selected fields, and data are presented as percent apoptotic cells. Apoptosis are verified by use western blot analysis of total and cleaved caspase3.

Tumor Volume Assessment. LLC, H450 and H23 cells are implanted into C57BL6 and nude mice, respectively. 106 viable cells suspended in 0.1 mL of cell medium are injected s.c. into the hind-limb. Each group of mice are comprised of 12 mice which are stratified into two groups to create approximately the same mean tumor volume. The mean volume of the tumors in mice at the time of treatment (day 0) with radiation, control, irradiated, LBH589 alone and LBH589 given prior to radiation are approximately 200 mm³. Irradiated mice are immobilized with 140 μL of ketamine, and the entire body are shielded with lead, except for the tumor-bearing hind limb. A total dose of 21 Gy are administered in seven fractionated doses on days 0-4, 7 and 8. The LBH589 group receive of LBH589 administered p.o. via esophageal injection on days-1 and 6.

### Treatment Groups.

| LBH589 | Radiation |
|---|---|
| 10 mg/kg 5×/wk | 3 Gy × 7 |
| 40 mg/kg 1×/wk | 3 Gy × 7 |
| 0 | 3 Gy × 7 |
| 10 mg/kg 5×/wk | 0 |
| 40 mg/kg 1×/wk | 0 |
| 0 | 0 |

Twelve nude mice implanted with H450 AND H23 cells in the same manner as described above for LLC. The mean volume of the tumors in mice at the time of treatment (day 0) with radiation in control, irradiated, LBH589 and LBH589 with radiation are 200 mm³.

Tumor volumes were measured on days 0, 2, 4, 7, 9, 11, 14, 16, 18, 20 and 22 using skin calipers. Tumor volumes were calculated from a formula (a×b×c/2) that are derived from the formula for an ellipsoid (πd3/6). Data were calculated as the percentage of original (day 0) tumor volume and graphed as fractional tumor volume±SD for each treatment group.

Tumor Histological Sections. C57BL6 mice are injected with 10⁶ LLC cells suspended in 0.1 mL of cell medium s.c. into the right hind-limb. Tumors are allowed to grow over a period of fourteen days. Three mice are treated with LBH589 and three mice are untreated controls. One hour after treatment mice are sacrificed and tumors are collected, fixed in formaldehyde and sectioned. Sections from LBH589 treated mice and controls are then probed for with phospho-Akt antibody (Cell Signaling 1:1000). TUNEL staining are performed as we have described.

Statistical Analysis. Statistical Analysis are performed using SPSS and R software to calculate p-values using the student's t-test and the standard deviation of individual data points.

Results. LBH589 sensitizes NSCLC to the cytotoxic effects of ionizing radiation. Clonogenic analysis of H460 cell lines was performed using LBH589 and 0-6 Gy. FIG. 1 shows the mean surviving fraction and standard error for each treatment group (n=3). Cells were suspended, counted with a hemocytometer, and plated at specific cell densities. Once attached, cells were treated with LBH589 for 1 hour or 18 hours followed by 0, 2, 4, or 6 Gy. Media was changed after IR and the cells were allowed to proliferate for 10 days. Shown is the average and standard error of the relative fractions of colonies (n=3).

Untreated control cells demonstrated substantial radioresistance, with 6 Gy resulting in only one log reduction in survival. Use of LBH589 1 hour and 18 hours prior to IR resulted in a synergistic decrease in colony survival compared to untreated cells as evident by an increase in the negative slope of the dose response curve. Treatment with LBH589 alone for 18 hours resulted in a significantly reduced plating efficiency while treatment for 1 hour alone had no reduced plating efficiency compared to the control. These data show that LBH589 enhances the cytotoxic effects of ionizing radiation in NSCLC cell lines.

LBH589 enhances radiation induced apoptosis. To study the effect of HDAC inhibition by LBH589 on apoptosis three in vitro experiments were performed. FIG. 2 show results from Annexin V-FITC/PI flow cytometry analysis of apoptosis. H23 and H460 cells lines were treated with 25 nM LBH589 for 18 hrs then irradiated with 3 Gy. Twenty (20) hours later, cells were harvested, stained with Annexin-FITC and PI, and analyzed by flow cytometry. Shown is the number of apoptotic cells and standard error (n=3) for each treatment condition. *P<0.05 compared to control.

Use of 25 nM LBH589 prior to 3 Gy significantly increased the number apoptotic cells from 7% to 30% for H23 cell line (P<0.001) and from 6 to 25% for H460 cell line (P=0.003) compared to control. Use of IR alone or LBH589 alone produced only a minimal increase and the effect of the combined treatment was greater than what would be predicted by an additive effect.

To confirm the ability of LBH589 to sensitize human lung cancer cell lines to radiation induced apoptosis, nuclear morphology studies were performed. FIG. 3 shows the mean percentage and standard error of pyknotic nuclei determined by DAPI staining. DAPI staining of cells treated with LBH589 and IR. Cells were subcultured onto slides and treated with 25 nM LBH589 for 18 hours followed by 3 Gy. Eighteen (18) hours later cells were fixed and stained with DAPI. Shown is the percentage of pyknotic nuclei and standard error (n=3) determined by manual counts from microscopy. *P<0.05 compared to control.

Use of 25 nM LBH589 18 hours prior to 3 Gy significantly increased the percentage of pyknotic nuclei to over 10% for H23 (P<0.001) and H460 (P=0.042) cell lines. Untreated H23 and H460 cells had less than 1% apoptotic nuclei, H23 and H460 cells treated with 3 Gy had 3% and 2% apoptotic nuclei, and H23 and H460 cells treated with LBH589 alone had 4% and 2% apoptotic nuclei, respectively.

Cleavage of caspase 3 was analyzed to verify the role of apoptosis in cells treated with LBH589 and radiation. Western blot analysis was performed on H23 and H460 whole cell lysates. FIG. 4 shows the Western immunoblots for cleaved caspase3 and actin. LBH589 induced caspase3 cleavage. H23 and H460 cells were treated with 25 nM LBH589 for 18 hours then irradiated with 3 Gy. Six hours later, protein was extracted, quantified, run in a 12% SDS-polyacrylamide gel, transferred, and probed with antibodies to cleaved caspase3 and actin. Shown are the immunoblots of caspase3, cleaved caspase3, and actin from H23 and H460 cell lines.

An increase in caspase3 cleavage was evident in both H23 and H460 cell lines following treatment with LBH589. Use of LBH589 prior to IR increased levels of caspase3 cleavage in H460 cells. This increase, however, was not as prominent in the H23 cell line.

LBH589 enhances tumor growth delay in vivo. H460 cells were injected into the hind limb of mice. After tumor formation the mice were treated with two oral doses of 40 mg LBH589 and/or five 3 Gy fractions over seven days. FIG. 5 shows the fold increase in tumor volume (A) and the tumor growth delay (B) for each treatment group. Use of LBH589 alone resulted in a modest but significant tumor growth delay of two days (P<0.001). IR alone delayed growth by approximately 4 days (P<0.001). Combined treatment significantly delayed tumor growth by approximately 20 days (P<0.001) indicating that HDAC inhibition enhances the effects of IR on NSCLC tumor growth. In addition, the mice receiving LBH589 showed minimal signs of toxicity during the course of the study as monitored by weight loss and mobility.

FIG. 5B shows the effect of LBH589 and ionizing radiation in the xenograft tumor model. H460 cells were injected in the hind limb of nude mice and allowed to grow for one week. The mice were divided into four groups: control, 3 Gy, LBH589 40 mg, LBH 40 mg+3 Gy. LBH589 was administered via oral gavage 1 hour prior to IR. The mice were treated with two doses of LBH589 and 5 fractions of 3 Gy over the first seven days. A, shown is the mean fold increase in tumor volume and standard error for each treatment group (n=5). B, shown is the mean tumor growth delay and standard error calculated using a 10-fold increase in tumor volume as reference.

LBH589 prolongs the duration of radiation induced γ-H2AX foci. Immunostaining was performed to study γ-H2AX foci present at DNA double strand breaks. FIG. 6A shows representative photographs of the H23 cell line treated with combinations of LBH589 and IR. The red staining of γ-H2AX foci and blue staining of the DAPI counterstain are shown. 3 Gy induced γ-H2AX foci as early as 30 minutes following treatment. These foci disappeared by 6 hrs in cell lines treated with IR alone. Use of LBH589 alone for 20 hours resulted in a modest increase in γ-H2AX foci. In comparison, LBH589 added 18 hours prior to IR prolonged the duration of γ-H2AX foci for up to 24 hours after IR (42 hours after LBH589 administration). Furthermore, γ-H2AX foci were seen at 18 hours and 24 hours after IR in cells undergoing apoptosis (arrows). Interestingly, no γ-H2AX foci were present in cells undergoing apoptosis following treatment with radiation without HDAC inhibition. Similar results were seen in the H460 cell line (Supplementary Figure S1).

FIG. 6B shows the number of γ-H2AX foci present 24 hrs after IR. LBH589 prolongs duration of γ-H2AX foci in irradiated lung cancer cells. H23 cell line received the indicated treatment of 25 nM LBH589 and/or 3 Gy. Anti-γ-H2AX antibody was used for immunostaining with rhodamine red labeled secondary antibody (red). Cells were counterstained with DAPI (blue). Shown are representative photographs of the H23 cells line (A) at the indicated time points after IR. Arrows point to apoptotic cells. B, shown is the mean and standard error of cells with γ-H2AX nuclear foci. *P<0.05 compared to control. Treatment with 3 Gy alone and LBH589 alone resulted in rapid resolution of γ-H2AX (<5% at 24 hours). Use of LBH589 prior to IR significantly delayed the resolution of γ-H2AX foci with 60% residual foci in both cell lines at 24 hours (P<0.001). The increased duration of γ-H2AX foci following treatment with LBH589 and IR indicates that HDAC inhibition disrupts the DNA repair process and this mechanism potentially sensitizes NSCLC to the cytotoxic effects of radiation.

HDAC4 nuclear translocation in irradiated lung cancer cell lines.

Immunostaining of HDAC4 was performed on H23 and H460 cell lines to identify the effect of LBH589 on HDAC4 compartmentalization. FIG. 7 shows representative photographs of the H460 cell line probed with anti-HDAC4 antibodies and rhodamine labeled secondary antibodies (red) then counterstained with DAPI (blue). Untreated cells and cells treated with LBH589 alone showed background HDAC4 staining in the cytoplasm and nucleus. When H460 cells were treated with 3 Gy, HDAC4 localized to the nucleus at 2 hours and minimal HDAC4 was present in the cytoplasm. However, LBH589 added prior to IR markedly limited HDAC4 nuclear localization. A similar effect was seen in the H23 cell line. These results were confirmed in the H460 cell line using anti-HDAC4 antibodies for Western blot analysis of cytoplasmic and nuclear proteins.

1. A method for treating a proliferative disease in a subject in need of such treatment, wherein the method comprises administering:
(a) N-hydroxy-3-[4-[[[2-(2-methyl-1H-indol-3-yl)-ethyl]-amino]methyl]phenyl]-2E-2-propenamide, of formula (III): 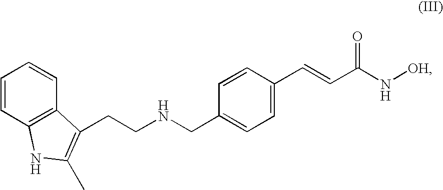
or a pharmaceutically acceptable salt thereof, in combination with
(b) ionizing radiation.

2. A method according to claim 1 wherein subject is a warm-blooded animal having a proliferative disease comprising administering to the animal a combination according to claim 1 in a way that is jointly therapeutically effective against a proliferative disease.

3. A method according to claim 1 which comprises administering a quantity which is jointly therapeutically effective against a proliferative disease of a compound of formula (III) and at least one pharmaceutically acceptable carrier for use in combination with ionizing radiation.

4. A method according to claim 1 for the delay of progression of a proliferative disease in a subject in need of such treatment.

5. A method according to claim 1 wherein the proliferative disease is a solid tumor.

6. A package comprising a compound of formula (III) in pharmaceutically acceptable form, together with instructions for the use in combination with ionizing radiation for the treatment of a proliferative disease.
